(19) United States
(12) Patent Application Publication
(10) Pub. No.: US 2009/0186896 A1
(43) Pub. Date: Jul. 23, 2009

(54) PDE Inhibitors and Combinations Thereof for the Treatment of Urological Disorders

(75) Inventors: Ernst Ulbrich, Eltville (DE); Peter Sandner, Wuppertal (DE); Hanna Tinel, Wuppertal (DE); Joachim Hütter, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/992,779

(22) PCT Filed: Sep. 16, 2006
(86) PCT No.: PCT/EP2006/009040

§ 371 Date: Mar. 18, 2009

(30) Foreign Application Priority Data
Sep. 29, 2005 (EP) 05021261.2
Apr. 13, 2006 (EP) 06007776.5

(51) Int. Cl. A61K 31/501; G01N 33/566; A61K 31/497; A61K 31/506; A61P 15/10

(52) U.S. Cl. 514/248; 436/501; 514/252.16; 514/250; 514/275

(57) ABSTRACT

The invention provides pharmacological compositions comprising PDE-5 and PDE-4 inhibitors, alone or in combination, for the treatment of urological disorders comprising Benign Prostate Hyperplasia (BPH), Lower Urinary Tract Symptoms (LUTS) and in particular irritative symptoms caused by BPH-induced bladder outlet obstruction (BOO). The invention also provides methods of screening for such PDE-5 and PDE-4 inhibitors for use, alone and in combination, in the preparation of medicaments for the treatment of said urological disorders.

### TECHNICAL FIELD OF THE INVENTION

The present invention relates to phosphodiesterases (PDEs) and the pharmacology of PDE inhibitors. More particularly, the invention relates to PDE-5 and PDE-4 inhibitors and their use for preparation of medicaments for the treatment of urological disorders.

### BACKGROUND OF THE INVENTION

Benign prostate hyperplasia (BPH) resulting in bladder outlet obstruction (BOO) is a very common neoplasm in men. It is estimated that approximately 80% of men older than 50 years have moderate to severe symptoms, including increased urinary frequency, nocturia and urgency, accompanied by a slow urinary stream and urinary retention. Therefore BPH is increasingly recognized as a major health care problem in westernized countries (Guess 1995). Besides prostatic surgery (20% of all BPH patients), the common treatment of the disease comprises 5-alpha reductase inhibitors (finasteride) and alpha blockers (tamsulosin, doxazosin, terazosin, alfuzosin) (Truss 2001). 5-alpha reductase inhibitors influence the mechanical component of BPH and inhibit proliferation of the prostate tissue. Alpha blockers influence the dynamic component and decrease the irritative symptoms of BPH via relaxation of the prostatic smooth muscle which decreases the urethral resistance. Moreover alpha-blockers are able to relax directly bladder smooth muscle cells and reduce the non voiding contractions of the bladder. However all these treatment options have limited efficacy and/or an unfavorable side effect profile (Carbone 2003). Thus, diverse attempts have focused on new therapeutic options to inhibit the proliferation of the prostatic stroma or to decrease the tone of the smooth muscle of the prostate and bladder. This includes i.e. aromatase inhibitors (Sciarra 2000), growth factor antagonists (Desgrandchamps 1997), potassium channel openers (Gopalakrishnan 2004), and endothelin antagonists (Andersson 2002).

It is also well established that the cyclic nucleotides cAMP and cGMP can reduce smooth muscle tone (Drescher 1994). cAMP and cGMP are synthesized from their corresponding nucleoside triphosphates by the adenylate and guanylate cyclase respectively. They are degraded by the cyclic nucleotide phosphodiesterases (PDEs) which regulate the intracellular cAMP and cGMP level very effectively. Up to now 11 different PDE family members have been identified which differ in structure, regulation and specificity for their substrate (Soderling 2000). The role of PDEs for the treatment of Urological Disorders is only poorly understood, the characterization of PDE isoforms has lagged behind other systems and much of the literature was published prior to identification of the newly identified PDEs. Although PDEs are expressed in the lower urinary tract i.e. in bladder, urethral and in prostate tissues, mRNA expression data and direct comparisons of all PDE isogenes are still missing or inconsistent. There are some evidences that unspecific PDE inhibition is able to relax human prostate tissue (Drescher 1994). The data about the effect of PDE-5 inhibition is very limited. It has been shown, that Zaprinast, a PDE-5 inhibitor which also inhibits PDE-6, -9 and -11 is able to relax pre-contracted human prostate tissue in vitro (Ückert 2001), whereas the role of other PDE families within this tissue needs to be determined. Within the bladder, unspecific blockade of different PDEs by IBMX (inhibition of PDE-1, -2, -3, -4, -5, -6, -10, and -11) could relax bladder of female Guinea Pigs whereas Zaprinast was ineffective (Gillespie 2004). Despite these inconsistent findings the role of PDE-5 in relaxation of the corpus cavernosum and the treatment of erectile dysfunction is well known and there are already very potent and selective PDE-5 inhibitors in the market. Potent and selective PDE-4 inhibitors are mainly used for the treatment of Asthma and COPD (Spina 2003).

### DISCLOSURE OF THE INVENTION

One aspect of the invention is provided by a PDE mRNA expression profile demonstrating the abundance of cGMP-dependent PDE-5 and cAMP-dependent PDE-4 not only in the prostate but also in bladder tissue (FIGS. 1, 2). Therefore, selective inhibitors of PDE-5 or PDE-4, and in particular combinations of both, should not only reduce prostate contractility but also, as an additional benefit of a combination of both, ameliorate irritative symptoms caused by bladder outlet obstruction as it frequently occurs in urological disorders. Selective inhibitors of PDE-5 are i.e. Vardenafil, Sildenafil and Tadalafil, a selective inhibitor of PDE-4 is i.e. Roflumilast.

Urological disorders addressed by therapeutic agents of the invention comprise Benign Prostate Hyperplasia (BPH), Lower Urinary Tract Symptoms (LUTS) and in particular irritative symptoms caused by BPH-induced bladder outlet obstruction (BOO). Because not only symptomatic irritations of the bladder but also underlying BPH-induced bladder outlet obstructions are addressed by treatment with specific PDE-5 and/or PDE-4 inhibitors (and in particular combinations thereof), this treatment provides substantial advantage over methods of treatment already known in the art.

Other urological disorders which in particular and with substantial advantage can be treated by the above mentioned inhibitors, or combination of inhibitors are genitourinary disorders comprising neurogenic bladder syndrome [also referred to as overactive bladder (OAB) or interstitial cystitis (IC)], urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction.

Another aspect of the invention is the demonstration that the PDE-5 inhibitor Vardenafil has a relaxing effect on rat urethral rings with an EC. % value of 0.96 μmol/l, and rat prostate and bladder strips with the EC₅₀ value of 1.1 and 5.0 μmol/l respectively (FIG. 3, Table 1).

Another aspect of the invention is the demonstration that the PDE-4 inhibitor Roflumilast and the PDE-5 inhibitor Vardenafil both show relaxing effects on rabbit bladder strips with an IC50 of 260 mmol/l and 1.7 μmol/l respectively (FIG. 4, Table 2).

Another aspect of the invention is provided by the demonstration that the PDE-5 inhibitor Vardenafil significantly decreased the number of non-voiding contractions as a measure of irritative symptoms of BPH in the rat bladder outlet obstruction (BOO) model (FIG. 5).

The invention provides PDE-5 inhibitors which are, alone or in combination with PDE-4 inhibitors, useful for the treatment of urological disorders. In particular, compounds of the invention are Tadalafil ((6R,12aR)-2,3,6,7,12,12a-Hexahydro-2-methyl-6-(3,4-methylene-dioxyphenyl)pyrazino(1′,2′:1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), Udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxyphenyl)methyl]amino}-2-[(2S)₂-(hydroxymethyl)pyrrolidin-1-yl]-N-pyrimidin-2-ylmethyl)pyrimidine-5-carboxamide, SLx 2101 of Surface Logix, LAS 34179 Triazolo[1,2-]xanthine, 6-methyl-4-propyl-2-[2-propoxy-5-(4-methylpiperazino)sulfonyl]phenyl-, Roflumilast (3-(cyclopropylmethoxy)-N-3,5-dichloropyridin-4-yl)-4-difluoromethoxy)benzamide), Cilomilast (4-cyano-4-(3 cyclopentoxy-4-methoxy-phenyl)-cyclohexane-1-carboxylic acid), and Piclamilast (3-cyclopentoxy-N-3,5-dichloropyridin-4-yl)-4-methoxy-benzamide).

Still another aspect of the invention is a method of screening for PDE inhibitors, in particular for inhibitors of PDE-4- and PDE-5 for use, alone or in combination, for the preparation of medicaments for the treatment of urological disorders mentioned above.

The invention provides methods (also referred to herein as “screening assays”) for identifying PDE inhibitors which can be used for the treatment of urological disorders. The methods entail the identification of candidate or test compounds or agents (e.g., peptides, peptidomimetics, small molecules or other molecules) which bind to phosphodiesterases and/or have a stimulatory or inhibitory effect on the biological activity of PDE1A or its expression and then determining which of these compounds have an effect on symptoms or diseases regarding urological disorders in an in vivo assay.

Candidate or test compounds or agents which bind to PDE-4 or PDE-5 and/or have a stimulatory or inhibitory effect on the activity or the expression of PDE-4 or PDE-5 are identified either in assays that employ cells which express PDE-4 and/or PDE-5 (cell-based assays) or in assays with isolated PDE-4 and/or PDE-5 (cell-free assays). The various assays can employ a variety of variants of PDEs (e.g., full-length PDEs, a biologically active fragment of PDEs, or a fusion protein which includes all or a portion of PDEs). Moreover, PDE-4 and/or PDE-5 can be derived from any suitable mammalian species. The assay can be a binding assay entailing direct or indirect measurement of the binding of a test compound or a known PDE-4 or PDE-5 ligand to PDE-4 or PDE-5. The assay can also be an activity assay entailing direct or indirect measurement of the activity of PDE-4 or PDE-5. The assay can also be an expression assay entailing direct or indirect measurement of the expression of PDE-4 and/or PDE-5 mRNA or PDE-4 and/or PDE-5 protein. The various screening assays are combined with an in vivo assay entailing measuring the effect of the test compound on the symptoms of urological disorders.

The present invention includes biochemical, cell free assays that allow the identification of inhibitors and agonists of PDEs suitable as lead structures for pharmacological drug development. Such assays involve contacting PDE-4 and/or PDE-5 with a test compound and determining the ability of the test compound to act as an antagonist (preferably) or an agonist of the enzymatic activity of PDE-4 and/or PDE-5. In one embodiment, the assay includes monitoring the PDE activity of PDE-4 and/or PDE-5 by measuring the conversion of either cAMP or cGMP to its nucleoside monophosphate after contacting PDE-4 and/or PDE-5 with a test compound.

For example, cAMP and cGMP levels can be measured by the use of the tritium containing compounds 3HcAMP and 3HcGMP as described in [Hansen, R. S., and Beavo, J. A., PITAS USA 1982,79: 2788-92]. To screen a compound pool comprised of a large number of compounds, the microtiter plate-based scintillation proximity assay (SPA) as described in [Bardelle, C. et al. (1999) Anal. Biochem. 275: 148-155] can be applied.

Alternatively, the phosphodiesterase activity of the recombinant protein can be assayed using a commercially available SPA kit (Amersham Pharmacia). The PDE enzyme hydrolyzes cyclic nucleotides, e.g. cAMP and cGMP to their linear counterparts. The SPA assay utilizes the tritiated cyclic nucleotides [3H]cAMP or [3H]cGMP, and is based upon the selective interaction of the tritiated non cyclic product with the SPA beads whereas the cyclic substrates are not effectively binding.

Radiolabelled product bound to the scintillation beads generates light that can be analyzed in a scintillation counter.

A pharmaceutical composition of the invention is formulated to be compatible with its intended route of administration. Examples of routes of administration include parenteral (e.g., intravenous, intraarterial, intradermal, subcutaneous, intramuscular, inhalative, transdermal, transmucosal, nasal and rectal administration), oral (e.g. buccal, sublingual, oral mucosal and peroral administration) and local (e.g. local instillation of solutions or suspensions and local implants)

Pharmaceutical compositions suitable for injections and infusions include sterile aqueous solutions (if the active ingredient is sufficiently soluble in water), suspensions, emulsions and sterile powders for the extemporaneous preparation of sterile injectable solutions or dispersions. The carrier can be a solvent or dispersion medium containing, for example, water, ethanol, a pharmaceutically acceptable polyol like glycerol, propylene glycol, liquid polyethylene glycol, and suitable mixtures thereof. Pharmaceutically acceptable ingredients may be added like buffers, preservatives, antioxidants, isotonizing agents or surfactants. Depot injections are based on known formulations principles like oily solutions or suspensions or particles of biodegradable polymers.

For administration by inhalation, the compounds are delivered in the form of an aerosol spray from a pressurized container or dispenser which contains a suitable propellant from a nebulizer or a dry powder inhaler.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, sublingual or buccal preparations or suppositories. For transdermal administration, the active compounds are formulated into ointments, patches, gels, or creams as generally known in the art.

The compounds can also be prepared in the form of suppositories (e.g., with conventional suppository bases such as cocoa butter and other glycerides) or retention enemas for rectal delivery.

Oral compositions generally include an inert diluent or bulking agent and functional excipients. They can be enclosed in capsules or compressed into tablets. Other suitable dosage forms are effervescent tablets, chewable tablets, orodispersible tablets, softgelatine capsules, liquid filled hardgelatine capsules, powders in sachets and oral liquids.

Suitable functional excipients for the preparation of oral dosage forms are well known in the art and include, for example, binders such as polyvinylpyrrolidone or hydroxypropylmethylcellulose, disintegrants such as crospovidone or croscarmellose sodium, glidants like colloidal silicum dioxide, lubricants such as magnesium stearate, macrogols or stearic acid, sweetening agents such as aspartame, sucrose or saccharin sodium and flavouring agents such as peppermint or orange flavouring.

In one embodiment, the active compounds are prepared with carriers that will protect the compound against rapid elimination from the body, such as controlled release tablets or coated pellets filled in capsules or parenteral controlled release formulations, including implants and microencapsulated delivery systems. Biodegradable or biocompatible polymers can be used, such as ethylene vinyl acetate, polyanhydrides, polyglycolic acid, collagen, polyorthoesters, polylactic acid or polyglycolic-polylactic-copolymers.

In another embodiment the invention provides combinations of PDE-4 and PDE-5 inhibitors and their use for the preparation of pharmaceutical compositions for the treatment of urological disorders, whereby these combinations comprise either i) pharmaceutical compositions comprising a compound having inhibitory action on both PDE-4 and PDE-5 activity, or ii) pharmaceutical compositions comprising at least one PDE-4 inhibitor and at least one PDE-5 inhibitor as a fixed combination in one application unit, or iii) a kit of parts containing at least two sets of pharmaceutical compositions, each set consisting of at least one pharmaceutical preparation comprising a PDE-5 inhibitor in units of at least one dose and at least one pharmaceutical preparation comprising a PDE-4 inhibitor in units of at least one dose, whereby each application unit of said pharmaceutical compositions is administered in combination, sequentially, as single dose or in multiple doses.

The present invention provides further:

A method of screening for PDE 5 inhibitors useful as therapeutic agents in the treatment of a disease comprised in a group of diseases consisting of Benign Prostate Hyperplasia (BPH), Bladder Outlet Obstruction (BOO) and Lower Urinary Tract Symptoms (LUTS) comprising the steps of i) contacting a test compound (which may or may not have PDE-4 inhibitory activity) with a PDE5 polypeptide, ii) determining the activity of the PDE5 polypeptide at a certain concentration of the test compound or in the absence of said test compound, iii) determining the activity of said PDE5 polypeptide at a different concentration of said test compound, iv) selecting at least one compound with inhibitory effect on the PDE-5 polypeptide.

A method of screening for PDE 4 inhibitors useful as therapeutic agents in the treatment of a disease comprised in a group of diseases consisting of Benign Prostate Hyperplasia (BPH), Bladder Outlet Obstruction (BOO) and Lower Urinary Tract Symptoms (LUTS) comprising the steps of i) contacting a test compound (which may or may not have PDE-5 inhibitory activity) with a PDE4 polypeptide, ii) determining the activity of the PDE4 polypeptide at a certain concentration of the test compound or in the absence of said test compound, iii) determining the activity of said PDE4 polypeptide at a different concentration of said test compound, iv) selecting at least one compound with inhibitory effect on the PDE-4 polypeptide.

A method of screening for combinations of PDE 5 inhibitors and PDE 4 inhibitors useful as therapeutic agents in the treatment of a disease comprised in a group of diseases consisting of Benign Prostate Hyperplasia (BPH), Bladder Outlet Obstruction (BOO) and Lower Urinary Tract Symptoms (LUTS) comprising the steps of i) contacting a first test compound with a PDE5 polypeptide, ii) determining the activity of the PDE5 polypeptide at a certain concentration of the first test compound or in the absence of said first test compound, iii) determining the activity of the PDE5 polypeptide at a different concentration of said first test compound, iv) selecting at least one first compound with inhibitory effect on the PDE-5 polypeptide, v) contacting a second test compound with a PDE4 polypeptide, vi) determining the activity of the PDE4 polypeptide at a certain concentration of the second test compound or in the absence of said second test compound, vii) determining the activity of the PDE4 polypeptide at a different concentration of said second test compound, vii) selecting at least one second compound with inhibitory effect on the PDE-4 polypeptide, vii) combining at least one first compound with PDE5 inhibitory activity with at least one second compound having PDE4 inhibitory activity.

Methods of screening which involve contacting the test compound in or at the surface of a cell, wherein the cell is in vitro.

Methods of screening which involve contacting the test compound with the PDE-4 or PDE-5 polypeptide in a cell free system.

Methods of screening may involve a test compound which is coupled to a detectable label.

In particular, the present invention provides:

A pharmaceutical composition for the treatment of a disease comprised in a group of diseases consisting of Benign Prostate Hyperplasia (BPH), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal, comprising a therapeutic agent which regulates the activity of a PDE5 polypeptide.

A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a therapeutic agent which regulates the activity of a PDE4 polypeptide.

A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a therapeutic agent which is a combination of the above mentioned selective therapeutic agents.

A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a therapeutic agent which regulates the activity of a PDE5 polypeptide and a PDE4 polypeptide.

A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a PDE-4 inhibitor selected from the group of PDE-4 Inhibitors consisting of Roflumilast (3-(cyclopropylmethoxy)-N-3,5-dichloropyridin-4-yl)-4-(difluoro-methoxy)benzamide), Cilomilast (4-cyano-4-(3-cyclopentoxy-4-methoxy-phenyl)-cyclohexane-1-carboxylic acid) and Piclamilast (3-cyclopentoxy-N-3,5-dichloropyridin-4-yl)-4-methoxy-benzamide).

A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal comprising a PDE-5 inhibitor selected from the group of PDE-5 Inhibitors consisting of Tadalafil ((6R,12aR)-2,3,6,7,12,12a-Hexahydro-2-methyl-6-(3,4-methylene-dioxyphenyl)pyrazino(1′,2′: 1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil(3-[2-ethoxy-5-(4-methylpiperazin-1-yl)sulfonyl-phenyl]-7-methyl-1-9-propyl-2,4,7,8-tetrazabicyclo[4.3.0]nona-3,8,10-trien-5-one), Udenafil 5-[2-propyloxy-5-(1-methyl-2-pyrrolidinyl-ethyl-amidosulfonyl)phenyl]-methyl-3-propyl-1,6 dihydro-7H-pyrazolo(4,3-d)pyrimidine-7-one, Dasantafil 7-(3-Bromo 4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)₂-(hydroxymethyl)pyrrolidin-1-yl]-N-(pyrimidin-2-ylmethyl)pyrimidine-5-carboxamide, SLx 2101 of Surface Logix, LAS 34179Triazolo[1,2-]xanthine, 6-methyl-4-propyl-2-[2-propoxy-5-(4-methylpiperazino)sulfonyl]phenyl or salts, hydrates or hydrates-f salts thereof.

A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a combination of at least one PDE-4 inhibitor selected from the group of PDE-4 inhibitors consisting of Roflumilast (3-cyclopropylmethoxy)-N-3,5-dichloropyridin-4-yl) 4-(difluoromethoxy) benzamide), Cilomilast (4-cyano-4-(3-cyclopentoxy-4-methoxy-phenyl)cyclohexane-1-carboxylic acid) and Piclamilast (3-cyclopentoxy-N-3,5-dichloropyridin-4-yl)-4-methoxy-benzamide) and at least one PDE-5 inhibitor selected from the group of PDE-5 inhibitors consisting of Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), and Tadalafil ((6R,12aR)-2,3,6,7,12,12a-Hexahydro-2-methyl-6-(3,4-methylene-dioxyphenyl).

Use of a PDE5 inhibitor for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UL) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

Use of a PDE4 inhibitor for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal.

Use of a combination of at least one PDE4 inhibitor and at least one PDE5 inhibitor for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction in a mammal.

Use of an agent which is a inhibitor of a PDE4 polypeptide and a PDE5 polypeptide for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal.

Use of PDE-5 inhibitor selected from the group of PDE-5 Inhibitors consisting of Tadalafil ((6R,12aR)-2,3,6,7,12,12a-Hexahydro-2-methyl-6-(3,4-methylene-dioxyphenyl) pyrazino(1′,2′: 1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methyl-1-9-propyl-2,4,7,8-tetrazabicyclo[4.3.0]nona-3,8,10-trien-5-one), Udenafil 5-[2-propyloxy-5-(1-methyl-2-pyrrolidinyl-ethyl-amidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)₂-(hydroxymethyl)pyrrolidin-1-yl]-N-(pyrimidin-2-ylmethyl)pyrimidine-5-carboxamide, SLx 2101 of Surface Logix, LAS 34179Triazolo[1,2-]xanthine, 6-methyl-4-propyl-2-[2-propoxy-5-(4-methylpiperazino)sulfonyl]phenyl or salts, hydrates or hydrates of salts thereof, for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

Use of PDE-4 inhibitor selected from the group of PDE-4 Inhibitors consisting of Roflumilast (3-cyclopropylmethoxy)-N-3,5-dichloropyridin-4-yl)-4-difluoromethoxy) benzamide), Cilomilast (4-cyano-4-(3-cyclopentoxy-4-methoxy-phenyl)cyclohexane-1-carboxylic acid) and Piclamilast (3-cyclopentoxy-N-3,5-dichloropyridin-4-yl)-4-methoxy-benzamide) for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal.

Use of a combination of at least one PDE-4 inhibitor selected from the group of PDE-4 inhibitors consisting of Roflumilast (3-cyclopropylmethoxy)N-(3,5-dichloropyridin-4-yl) 4-difluoromethoxy)benzamide), Cilomilast (4-cyano-4-(3-cyclopentoxy-4-methoxy-phenyl)cyclohexane-1-carboxylic acid) and Piclamilast (3-cyclopentoxy-N-(3,5-dichloropyridin-4-yl)-4-methoxy-benzamide) and at least one PDE-5 inhibitor selected from the group of PDE-5 inhibitors consisting of Vardenafil (2-(2-Ethoxy-5-(4-ethylpiperazin-1-yl-1-sulfonyl)phenyl)-5-methyl-7-propyl-3H-imidazo (5,1-f) (1,2,4) triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), and Tadalafil ((6R,12aR)-2,3,6,7,12,12a-Hexahydro-2-methyl-6-(3,4-methylene-dioxyphenyl) for the preparation of a pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction in a mammal.

A Method for the preparation of a pharmaceutical composition useful for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction in a mammal comprising the steps of i) identifying a inhibitor of PDE5 according to the method of screening described above ii) identifying a inhibitor of PDE4 according to the method of screening described above) determining whether said inhibitors ameliorate the symptoms of a disease comprised in a group of diseases consisting of Benign Prostate Hyperplasia (BPH), Bladder Outlet Obstruction (BOO) and Lower Urinary Tract Symptoms (LUTS) in a mammal; and iii) combining at least one of said inhibitors with an acceptable pharmaceutical carrier.

A method for the preparation of a pharmaceutical composition wherein the inhibitor of PDE5 is a PDE-5 inhibitor selected from the group of PDE-5 Inhibitors consisting of Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), Tadalafil ((6R,12aR)-2,3,6,7,12,12a-Hexahydro-2-methyl-6-(3,4-methylenedioxyphenyl), Udenafil 5-[2-propyloxy-5-(1-methyl-2-pyrrolidinyl-ethyl-amidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S-2-hydroxymethyl)pyrrolidin-1-yl]-N-(pyrimidin-2-yl methyl)pyrimidine-5-carboxamide, SLx 2101 of Surface Logix and LAS 34179-Triazolo[1,2-]xanthine, 6-methyl-4-propyl-2-[2-propoxy-5-(4-methylpiperazino)-sulfonyl]phenyl.

A method for the preparation of a pharmaceutical composition wherein the inhibitor of PDE4 is a PDE4 inhibitor selected from the group of PDE4 Inhibitors consisting of Roflumilast (3-cyclopropylmethoxy)-N-3,5-dichloropyridin-4-yl)-4-difluoromethoxy) benzamide), Cilomilast (4-cyano-4-A3-cyclopentoxy-4-methoxy-phenyl)cyclohexane-1-carboxylic acid) and Piclamilast (3-cyclopentoxy-N-3,5-dichloropyridin-4-yl)-4-methoxy-benzamide).

Use of a pharmaceutical composition as mentioned above for the regulation of PDE activity in a mammal having a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction.

A kit of parts for the treatment of a disease comprised in a group of diseases consisting of Benign Prostate Hyperplasia (BPH), Bladder Outlet Obstruction (BOO) and Lower Urinary Tract Symptoms (LUTS) in a mammal including humans containing a combination of at least one pharmaceutical composition selected from the group of pharmaceutical compositions consisting of Vardenafil, Sildenafil and Tadalafil and at least one pharmaceutical composition selected from the group of pharmaceutical compositions consisting of Roflumilast, Cilomilast and Piclamilast.

A Method for the preparation of a kit of parts useful for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising the steps of i) selecting at least one pharmaceutical composition from the group of pharmaceutical compositions consisting of Vardenafil, Sildenafil and Tadalafil ii) selecting at least one pharmaceutical composition from the group of pharmaceutical compositions consisting of Roflumilast, Cilomilast and Piclamilast, iii) combining at least two of said pharmaceutical compositions thereby creating said kit of parts.

A kit of parts for the treatment of a disease comprised in a group of diseases consisting of Benign Prostate Hyperplasia (BPH), Bladder Outlet Obstruction (BOO) and Lower Urinary Tract Symptoms (LUTS) in a mammal containing a combination of at least one therapeutic agent regulating the activity of a PDE-5 polypeptide and Tadalafil and at least one therapeutic agent regulating the activity of a PDE-4 polypeptide.

A Method for the preparation of a kit of parts useful for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising the steps of i) selecting at least one pharmaceutical composition comprising a therapeutic agent regulating the activity of a PDE-5 polypeptide, ii) selecting at least one pharmaceutical composition comprising a therapeutic agent regulating the activity of a PDE-4 polypeptide, iii) combining at least two of said pharmaceutical compositions thereby creating said kit of parts.

A preferred embodiment of the invention is a pharmaceutical composition containing Vardenafil, or a salt, a hydrat or a hydrat of a salt thereof, for the treatment of a disease comprised in a group of diseases consisting of Benign Prostate Hyperplasia (BPH), Bladder Outlet Obstruction (BOO), Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy and obstructive uropathies in a mammal.

It has surprisingly be found that especially Vardenafil, or a salt, a hydrat or a hydrat of a salt thereof, exhibit higher activities and shows better results in the treatment of neurogenic bladder (also referred to as overactive bladder or interstitial cystitis) compared with other PDE-5 inhibitors.

### DESCRIPTION OF FIGURES

FIG. 1: Relative mRNA Expression of PDE-5 in Kidney (K), Bladder (B), Prostate (P), Urethra (U) and Corpus Cavernosum (C) of Sprague Dawley Rats. Data are mean+SEM, n=10.

FIG. 2: Relative mRNA Expression of PDE-4a, -4b, -4c, -4d and PDE-5 in bladder and prostate of Sprague Dawley Rats. Data are mean+SEM, n=10.

FIG. 3: Effects of Vardenafil on the contraction of isolated rat urethral rings (black triangles), and bladder- (black diamonds) and prostate strips (grey squares). The bladder strips were pre-contracted using K⁺ (50 mmol/l) Krebs-Henseleit solution. Prostate and urethral tissues were pre-contracted using 10 μmol/l Phenylephrine. The relaxation was expressed as percentage of the pre-contraction. Each point represents a mean value±SEM. n=9.

FIG. 4: Effects of Roflumilast (black diamonds) and Vardenafil (grey squares) on the contraction of isolated rabbit bladder strips. The bladder strips were pre-contracted using K⁺ (50 mmol/l) Krebs-Henseleit solution. The relaxation was expressed as percentage of the pre-contraction. Each point represents a mean value±SEM. n=9.

FIG. 5: Number of non-voiding contractions in % after bolus i.v. treatment with vehicle (V) and Vardenafil HCl (1, 3 and 10 mg/kg). Data are mean+SEM, *=significant with p<0.05 (paired student's t-test).

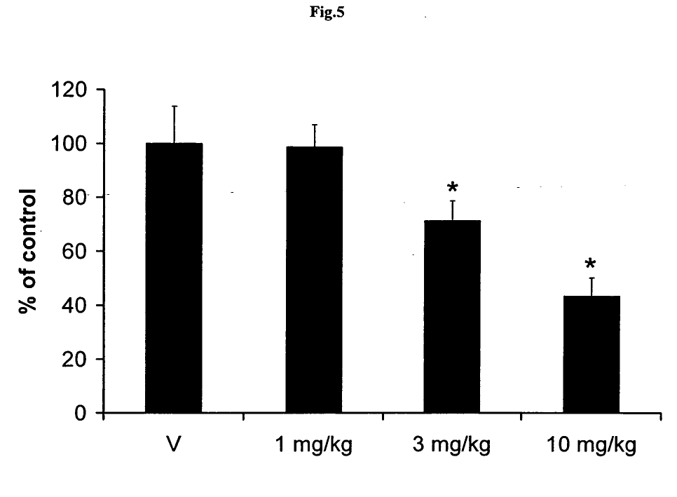

FIG. 6: Micturition interval in % after bolus i.v. treatment of vehicle (V) and Vardenafil HCl (1.3 mg/kg) compared to the basal micturation interval (C). Data are mean+SEM, *=significant with p<0.05 (paired student's t-test).

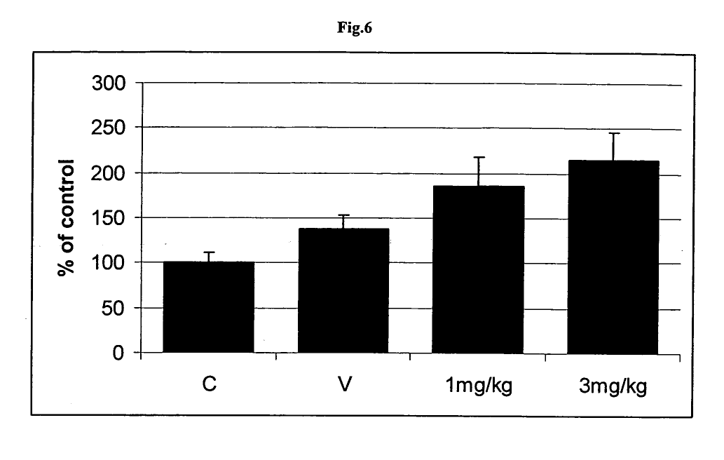

### EXAMPLE 1

Tissue sampling and RNA preparation: Male Sprague Dawley rats with a body weight between 200-250 g were used for tissue collecting. The rats were briefly anaesthetized with a mixture of 5% Isoflurane (Baxter S.A.) in a carrier with 70% N₂O and 30% O₂, and than euthanized by decapitation. The abdomen was opened by a midline incision and the kidneys and lower urinary tract tissue as renal medulla, urether, bladder prostate and urethra were exposed and quickly removed. The tissues were frozen in liquid N2 and stored until RNA preparation. Total RNA was isolated using RNeasy mini columns (Qiagen Inc.) and further purified by DNase digestion.

PDE mRNA quantification: The mRNA expression of the different PDE isogenes in rat lower urinary rat tissues was measured by real time quantitative PCR (TaqMan-PCR, Heid 1996). Therefore 1 μg of total RNA were transcribed into cDNA with Superscript 11 RT cDNA synthesis kit (Gibco, Inc) according to the manual of the supplier. The mRNA for the PDEs were measured by real-time quantitative RT-PCR on an ABI Prism 7700 sequence detection instrument (Applied Biosystems, Inc.). Specific Sequences for forward and reverse primers as for the fluorogenic probe of each PDE isogene mRNA were designed by Primer Express 1.5 Software (Applied Biosystems, Inc.). During PCR amplification, 5′ nucleolytic activity of Taq polymerase cleaves the probe separating the 5′ reporter fluorescent dye from the 3′ quencher dye. The threshold cycle, Ct, which correlates inversely with the target mRNA level, was measured as the cycle number at which the reporter fluorescent emission increases 10 standard deviations above background level. As housekeeping gene, beta-action was quantified as described above, using as forward primer 5′-accttcaacaccccagcca-3′, reverse primer 5′-cagtggtacgaccagaggca-3′ and fluorescent probe 5′-6AFM-acgtagccatccaggctgtgttgtcc-TAMARA-3′. The PDE mRNA levels were corrected for beta-actin mRNA levels and calculated as relative expression using comparative Ct-method.

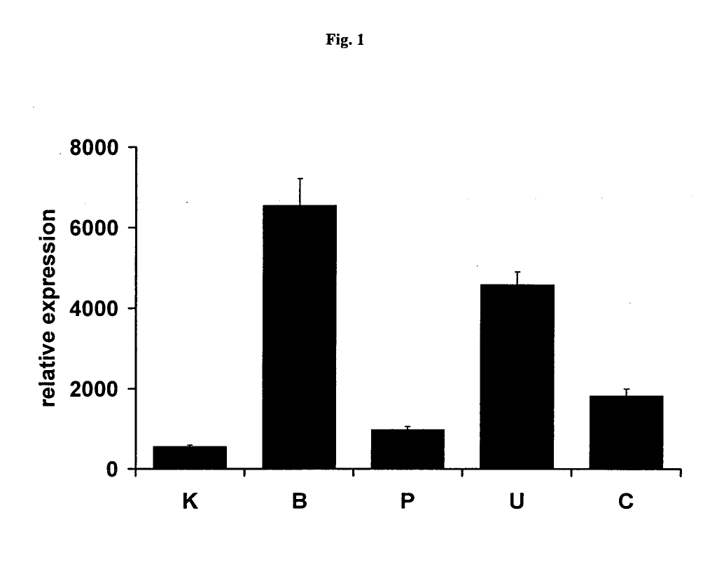

Expression of PDE-5 and PDE-4A, -4B, -4C, 4D mRNA in the lower urinary tract: Since there is only incomplete data on the expression profile of PDE-5 in lower urinary tract tissue, PDE mRNA was quantified in male Sprague Dawley rats via TaqMan RT-PCR. The most prominent expression of PDE-5 was found in the bladder (FIG. 1). Lower expression levels were found in the urethra, the corpus cavernosum and the prostate (FIG. 1). These result showed that there is a substantial PDF-5 mRNA expression in lower urinary tract tissues especially in the bladder.

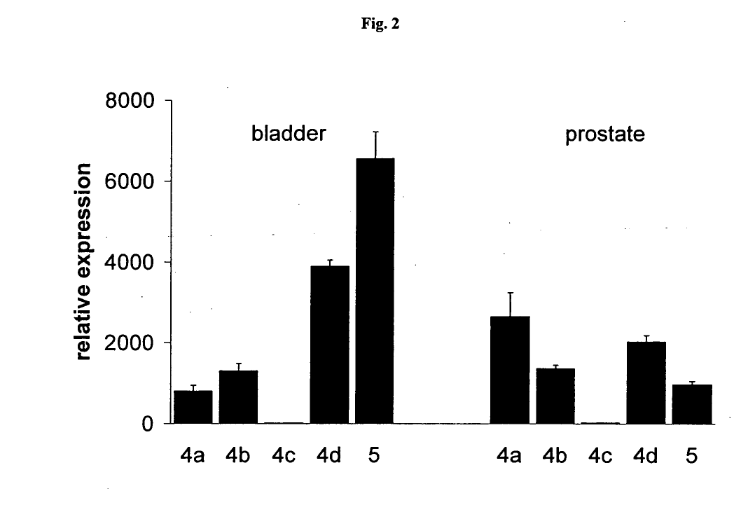

Moreover PDE-4 mRNA expression of all four PDE-4 isogenes (PDE-4A, -4B, -4C and -4D) was determined by TaqMan RT-PCR in the bladder and the prostate (FIG. 2). We found very low expression of PDE-4c, which was on the borderline of detectability, however PDE-4A, -4B and -4D mRNAs were substantially expressed within both tissues. In the bladder PDE-4D was the most abundant PDE-4 isogene mRNA whereas in the prostate PDE-4a and -4D are almost equally distributed and 2.5-fold higher expressed than PDE-5 mRNA (FIG. 2).

The expression profile demonstrates that PDE-5 and PDE-4D mRNAs are abundant in the bladder but also in prostate tissue. Therefore, inhibitors of PDE-5 or PDE-4, but in particular combinations of both, PDE-5 and PDE-4 inhibitors, such as Vardenafil in combination with Roflumoilast should not only reduce bladder but also prostate contractility, thus providing advantage over methods of treatment for urological disorders already existing in the state of the art, said disorders comprising i.e. Benign Prostate Hyperplasia (BPH) and in particular irritative symptoms caused by BPH-induced bladder outlet obstruction (BOO) including, but not limited to, Lower Urinary Tract Symptoms (LUTS).

### EXAMPLE 2

Tissue preparation: Male Wistar rats (200-300 g) were euthanized using carbon dioxide. The tissues were removed and placed in ice-cold Krebs-Henseleit buffer of following composition (in mmol/l): NaCl 112, KCl 5.9, CaCl₂ 2.0 MgCl₂ 1.2, NaH₂PO₄ 1.2, NaHCO₃ 25, glucose 11.5. Four equally sized longitudinal strips of approximately 2 mm×10 mm were cut from the bladder body. Prostate strips were obtained by cutting transversally through the lobes of the prostate gland parallel to the urethra. One ring per animal was dissected from the proximal part of the urethra.

White New Zealand rabbits were anesthetized using thiopental. The urinary bladder was removed and placed in ice-cold Krebs-Henseleit buffer of following composition (in mmol/l): NaCl 112, KCl 5.9, CaCl₂ 2.0 MgCl₂ 1.2, NaH₂PO₄ 1.2, NaHCO₃ 25, and glucose 11.5. Equally sized longitudinal strips of approximately 2 mm×10 mm were cut from the bladder body.

Recording of mechanical activity: The preparations were transferred to 20 ml organ baths containing Krebs-Henseleit solution equilibrated with 95% O₂, 5% CO₂ at 37° C. The strips were mounted between two hooks by means of two clips. For recording of isometric tension one of the hooks was connected to a force transducer which was in turn linked to an amplifier and chart recorder. The other hook was attached to a movable unit, permitting precise adjustment of preload tension. All tissues were then given a 60 min equilibration period during which they were washed and the resting tension was adjusted to 1 g every 20 min.

After the equilibration period, each experiment was started by exposing the preparation to K⁺ (50 mmol/l) Krebs-Henseleit solution. The procedure was repeated 3 times and the tissues were washed at least tree times between each contraction.

The bladder strips were than pre-contracted using K⁺ (50 mmol/l) Krebs-Henseleit solution. When the contraction was stabilized, an accumulative dose response curve of the compound tested was constructed. The stabilized contraction induced by K⁺ (50 mmol/l) Krebs-Henseleit solution was defined as 100% tension. The relaxation was expressed as percentage tension.

Prostatic strips and urethral rings were pre-contracted using 10⁻⁶ mol/l phenylephrine. The effects of the compounds on the prostate tissue were tested in a non-cumulative manner with washing steps between each concentration.

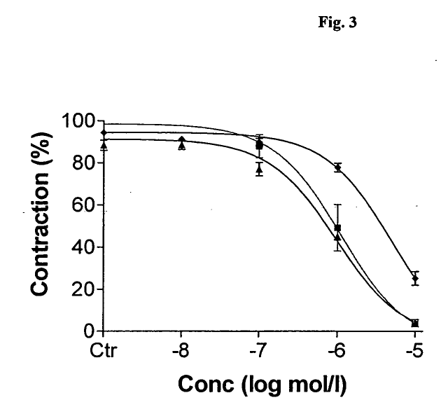

Organ bath assay: effects of Vardenafil on isolated rat urogenital organs: The effects of the PDE5 inhibitor Vardenafil on the relaxation of smooth muscles were tested in the organ bath system. The compound was applied in the concentration range from 10⁻⁸ mol/l to 10⁻⁵ mol/l (FIG. 3, Table 1). Vardenafil relaxed the urethral rings with an EC₅₀ value of 0.96 μmol/l, and the prostate and bladder strips with the EC₅₀ value of 1.1 and 5.0 μmol/l respectively.

TABLE 1
Effects of vardenafil on the contraction
of isolated rat urogenital tissues.

| Concentration | Bladder | Urethra | Prostate |
|---|---|---|---|
| (μmol/l) | (% contraction) | (% contraction) | (% contraction) |
| 0.001 | 94.3 ± 0.9 | 88.4 ± 2.4 | — |
| 0.01 | 91.2 ± 1.3 | 88.5 ± 2.1 | — |
| 0.1 | 89.9 ± 1.8 | 77.1 ± 3.9 | 99.2 ± 3.0 |
| 1 | 77.8 ± 2.1 | 44.9 ± 1.5 | 76.0 ± 2.3 |
| 10 | 25.3 ± 3.2 | 3.5 ± 1.7 | 25.3 ± 3.2 |

The relaxation is expressed as percentage of the pre-contraction. Each point represents a mean value ± SEM. n = 9.

### EXAMPLE 3

Tissue preparation: Male Wistar rats (200-300 g) were euthanized using carbon dioxide. The tissues were removed and placed in ice-cold Krebs-Henseleit buffer of following composition (in mmol/l): NaCl 112, KCl 5.9, CaCl₂ 2.0 MgCl₂ 1.2, NaH₂PO₄ 1.2, NaHCO₃ 25, glucose 11.5. Four equally sized longitudinal strips of approximately 2 mm×10 mm were cut from the bladder body. Prostate strips were obtained by cutting transversally through the lobes of the prostate gland parallel to the urethra. One ring per animal was dissected from the proximal part of the urethra.

White New Zealand rabbits were anesthetized using thiopental. The urinary bladder was removed and placed in ice-cold Krebs-Henseleit buffer of following composition (in mmol/l): NaCl 112, KCl 5.9, CaCl₂ 2.0 MgCl₂ 1.2, NaH₂PO₄ 1.2, NaHCO₃ 25, and glucose 11.5. Equally sized longitudinal strips of approximately 2 mm×10 mm were cut from the bladder body.

Recording of mechanical activity: The preparations were transferred to 20 ml organ baths containing Krebs-Henseleit solution equilibrated with 95% O₂, 5% CO₂ at 37° C. The strips were mounted between two hooks by means of two clips. For recording of isometric tension one of the hooks was connected to a force transducer which was in turn linked to an amplifier and chart recorder. The other hook was attached to a movable unit, permitting precise adjustment of preload tension. All tissues were then given a 60 min equilibration period during which they were washed and the resting tension was adjusted to 1 g every 20 min.

After the equilibration period, each experiment was started by exposing the preparation to K⁺ (50 mmol/l) Krebs-Henseleit solution. The procedure was repeated 3 times and the tissues were washed at least tree times between each contraction.

The bladder strips were than pre-contracted using K⁺ (50 mmol/l) Krebs-Henseleit solution. When the contraction was stabilized, an accumulative dose response curve of the compound tested was constructed. The stabilized contraction induced by K⁺ (50 mmol/l) Krebs-Henseleit solution was defined as 100% tension. The relaxation was expressed as percentage tension.

Prostatic strips and urethral rings were pre-contracted using 10⁻⁶ mol/l phenylephrine. The effects of the compounds on the prostate tissue were tested in a non-cumulative manner with washing steps between each concentration.

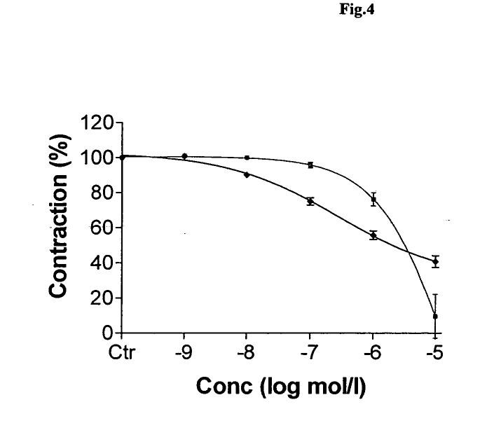

Effects of PDE5 and PDE4 inhibitors on isolated rabbit bladder strips: The effects of the PDE5 inhibitor vardenafil and the PDE4 inhibitor Roflumilast on the relaxation the bladder smooth muscle was tested in the organ bath using rabbit bladder strips. Both compounds were tested in the concentration from 10⁻⁹ mol/l to 10⁻⁵ mol/l (FIG. 4, Tab. 2). Roflumilast and vardenafil both relaxed the bladder strips with an IC50 of 260 nmol/l and 1.7 μmol/l respectively. (Table 2)

TABLE 2
Effects of Roflumilast and Vardenafil on the contraction
of isolated rabbit bladder strips.

| Concentration | Roflumilast | Vardenafil |
|---|---|---|
| (μmol/l) | (% contraction) | (% contraction) |
| 0.001 | 100.8 ± 0.6 | 100.9 ± 1.0 |
| 0.01 | 90.1 ± 1.7 | 99.9 ± 0.9 |
| 0.1 | 75.1 ± 2.1 | 95.7 ± 1.7 |
| 1 | 55.8 ± 2.5 | 76.3 ± 3.7 |
| 10 | 40.9 ± 3.3 | 9.6 ± 12.6 |

The relaxation is expressed as percentage of the pre-contraction. Each point represents a mean value ± SEM. n = 9.

### EXAMPLE 4

All animal experiments were performed due to the “German Law for the Protection of Laboratory animals” and were conducted due to the approved guidelines of the permission “Tierversuchsvorhaben No 401/A01 M010/M011 vom Sep. 7, 2004”. Experiments were performed with female Sprague Dawley Rats with a body weight between 200-250 g.

Bladder Outlet Obstruction: For the bladder outlet obstruction, rats were anesthetized with a mixture of 1.5-2% isoflurane in a carrier of 66% N₂O 33% O₂. The abdomen was shaved, opened by a lower midline incision, bladder and urethra were identified and the urethravesical junction was exposed. A 1.0 mm metal rod was placed along the proximal urethra and a 6-0 nylon ligature was tied tightly around the urethra and the rod. The rod was consecutively removed and the abdomen was closed by a silk ligature and cleaned up by 70% ethanol. There was a postoperative anti-pain treatment with 10 mg/kg Rimadyl® (Pfizer). Rats were kept then for 2 weeks and feed with tap water and standard rat chow. 24 hours prior to the cystometry rats were anaesthetized with isoflurane as described above. The laparotomy was performed as described above, the bladder was exposed and a polyethylene catheter (PE50) was implanted into the bladder dome. The catheter was tunneled subcutaneously using a cannula to reach the back neck of the animal. Additionally a catheter for intravenous administration (PE10) was placed into the jugular vein and tunneled subcutaneously to the back neck of the animal. Both catheters were fixed by a suture and a tape.

Conscious cystometry: For cystometry the animals were shortly anaesthetized by isoflurane as described above, placed in a Ballman's cage and fixed. Then animals were recovered at least for 1 h before the experiment started. The bladder catheter was then connected to the t-shaped tube to connect a pressure transducer for measurement of intra-bladder pressure (MLT0698, ADInstruments) and an infusion pump (Perfusor Compact®, Braun Melsungen) for continuous infusion of saline solution at a flow rate of 10 ml/h. The BOO animals showed an increase in bladder capacity (due to the bladder enlargement) and non voiding contractions (mimicking the irritative symptoms of BPH), when compared with a control animal. The efficacy of treatment was quantified via calculation of the non voiding contractions per micturition interval before and after treatment. For positive control the alpha receptor antagonist tamsulosin (10 μg/kg) was used. Values were given in % reduction of non voiding contractions.

Statistical analysis of results: Data are expressed as means±standard error of the means (SEM), and n indicating the number of experiments. The significance of differences between means was determined by paired and unpaired Student's t-test. Probability levels less than 0.05 were considered significant.

Effect of vardenafil on non voiding contractions in BOO rats: For the BOO model a partial ligature of the urethra was performed in rat under anesthesia. The bladder outlet obstruction (BOO) resulting from this procedure caused a significant increase of bladder weight (data not shown) indicating a pronounced bladder hypertrophy. It also caused non voiding contractions (NVC) of the bladder which were detected via cystometry in conscious animals. These NVC were a measure of irritative symptoms in BPH and were significantly reduced with an MED of 3 mg/kg Vardenafil i.v.

### EXAMPLE 5

All animal experiments were performed due to the “German Law for the Protection of Laboratory animals” and were conducted due to the approved guidelines of the permission “Tierversuchsvorhaben No 401/A01 M010/M011 vom Sep. 7, 2004”. Experiments were performed with female Sprague Dawley Rats with a body weight between 200-250 g. Anaesthetized cystometry: For cystometry female SD rats were anaesthetized with urethane (1.2 g/kg, ip). After laparotomy, the bladder was exposed and both ureters were ligated and cut. A polyethylene cannula (PE50) was implanted into the bladder dome, and the abdomen was closed. The bladder catheter was connected to the t-shaped tube to connect a infusion pump (Perfusor® compact; Braun Melsungen) for continuous infusion of saline solution and to connect a pressure transducer (Combitrans; Braun Melsungen) for measurement of intrabladder pressure. The intrabladder pressure signals were registered with the Powerlab System (MLT0698, ADInstrument). Cystometry was performed after 1 hr equilibration period from the surgical procedure. For i.v. drug treatment, the left femoral vein was cannulated with a polyethylene catheter. The effect of treatments was calculated on the micturition interval (corresponding to bladder capacity).

Induction of an overactive bladder was performed with 0.2% acetic acid solution (diluted with saline) infusion into the bladder instead of saline solution or with i.p. injection of 150 mg/kg of cyclophosphamide 18 hr before cystometry.

Statistical analysis of results: Data are expressed as means i standard error of the means (SEM), and n indicating the number of experiments. The significance of differences between means was determined by paired and unpaired Student's t-test. Probability levels less than 0.05 were considered significant.

Effect of vardenafil on micturition interval in CYP-treated rats: The micturition interval was significantly increased with an MED of 3 mg/kg Vardenafil i.v.

### REFERENCES

Andersson K E, Chapple C R, Hofner K. Future drugs for the treatment of benign prostatic hyperplasia. World J Urol 2002; 19:436-42.
Bardelle, C. et al. (1999) Anal. Biochem. 275: 148-155
Carbone D J Jr, Hodges S: Medical therapy for benign prostatic hyperplasia: sexual dysfunction and impact on quality of life. Int J Impot Res 2003; 15:299-306.
Desgrandchamps F. Clinical relevance of growth factor antagonists in the treatment of benign prostatic hyperplasia. Eur Urol. 1997; 32:28-31.
Drescher P, Eckert R E, Madsen P O. Smooth muscle contractility in prostatic hyperplasia: role of cyclic adenosine monophosphate. Prostate 1994; 25:76.
Gopalakrishnan M, Shieh C C. Potassium channel subtypes as molecular targets for overactive bladder and other urological disorders. Expert Opin Ther Targets 2004; 8:437-58.

Gillespie J I, Drake M J. Phosphodiesterase-linked inhibition of nonmicturition activity in the isolated bladder. BJU International 2004; 93:1325-1332
Guess H A, Epidemiology and natural history of benign prostatic hyperplasia. Urol Clin North Am 1995; 22:247.
Hansen, R. S., and Beavo, J. A., PITAS USA 1982,79: 2788-92
Heid C A, Stevens J, Livak K J, Williams P M, Real time quantitative PCR. Genome Res 1996; 6:986-994.
Sciarra F, Toscano V. Role of estrogens in human benign prostatic hyperplasia. Arch Androl 2000; 44:213-20.
Soderling S H, Beavo J A, Regulation of cAMP and cGMP signaling: new phosphodiesterases and new functions. Curr Opin Cell Biol. 2000; 12:174-179.
Spina D, Phosphodiesterase-4 Inhibitors in the treatment of inflammatory lung disease, Drugs 2003; 63:2575-2594
Truss M. C, Stief C, Machtens S, Wagner T, Jonas U (Eds) Pharmakotherapie in der Urologie, Springer 2001
Uckert S, Kuthe A, Jonas U, Stief C G, Characterization and functional relevance of cyclic nucleotide phosphodiesterase isoenzymes of the human prostate. J Urol 2001; 166:2484-2490.
Ying J, Yao D, Jiang Y, Ren X, Xu M, The positive effect of sildenafil on LUTS from BPH while treating E D Zhonghua Nan Ke Xue 2004 10:681-683.

1. A method of screening for PDE 5 inhibitors useful as therapeutic agents in the treatment of a disease comprised in a group of diseases consisting of Benign Prostate Hyperplasia (BPH), Bladder Outlet Obstruction (BOO) and Lower Urinary Tract Symptoms (LUTS) comprising the steps of i) contacting a test compound with a PDE5 polypeptide, ii) determining the activity of the PDE5 polypeptide at a certain concentration of the test compound or in the absence of said test compound, iii) determining the activity of said PDE5 polypeptide at a different concentration of said test compound, iv) selecting at least one compound with inhibitory effect on the PDE-5 polypeptide.

2. A method of screening for PDE 4 inhibitors useful as therapeutic agents in the treatment of a disease comprised in a group of diseases consisting of Benign Prostate Hyperplasia (BPH), Bladder Outlet Obstruction (BOO) and Lower Urinary Tract Symptoms (LUTS) comprising the steps of i) contacting a test compound with a PDE4 polypeptide, ii) determining the activity of the PDE4 polypeptide at a certain concentration of the test compound or in the absence of said test compound, iii) determining the activity of said PDE4 polypeptide at a different concentration of said test compound, iv) selecting at least one compound with inhibitory effect on the PDE-4 polypeptide.

3. A method of screening for combinations of PDE 5 inhibitors and PDE 4 inhibitors useful as therapeutic agents in the treatment of a disease comprised in a group of diseases consisting of Benign Prostate Hyperplasia (BPH), Bladder Outlet Obstruction (BOO) and Lower Urinary Tract Symptoms (LUTS) comprising the steps of i) contacting a first test compound with a PDE5 polypeptide, ii) determining the activity of the PDE5 polypeptide at a certain concentration of the first test compound or in the absence of said first test compound, iii) determining the activity of the PDE5 polypeptide at a different concentration of said first test compound, iv) selecting at least one first compound with inhibitory effect on the PDE-5 polypeptide, v) contacting a second test compound with a PDE4 polypeptide, vi) determining the activity of the PDE4 polypeptide at a certain concentration of the second test compound or in the absence of said second test compound, vii) determining the activity of the PDE4 polypeptide at a different concentration of said second test compound, vii) selecting at least one second compound with inhibitory effect on the PDE-4 polypeptide, vii) combining at least one compound with PDE5 inhibitory activity with at least one compound having PDE4 inhibitory activity.

4. A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI) like mixed-, urge-, stress-, or overflow incontinence (MUI, UUI, SUI, OUI), pelvic pain, benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies and erectile dysfunction in a mammal comprising a therapeutic agent which regulates the activity of a PDE5 polypeptide.

5. A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a therapeutic agent which regulates the activity of a PDE4 polypeptide.

6. A pharmaceutical composition for the treatment of a disease comprised in a group of diseases consisting of Benign Prostate Hyperplasia (BPH), Bladder Outlet Obstruction (BOO) and Lower Urinary Tract Symptoms (LUTS) in a mammal comprising a therapeutic agent which is a combination of the therapeutic agents of claims 4 and 5.

7. A pharmaceutical composition for the treatment of a disease selected from the group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI), pelvic pain, benign and malign disorders of the organs constituting the genitourinary system of female and male, renal diseases, immunologically mediated renal diseases, lupus nephritis, immune complex renal diseases, glomerulopathies, nephritis, toxic nephropathy, obstructive uropathies and erectile dysfunction in a mammal comprising a PDE-5 inhibitor selected from the group of PDE-5 Inhibitors consisting of Vardenafil, Sildenafil, Tadalafil, Udenafil, Dasantafil, Avanafil, SLx2101 and LAS34179.

8. A method for the treatment of a disease selected from the group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI), pelvic pain, benign and malign disorders of the organs constituting the genitourinary system of female and male, renal diseases, immunologically mediated renal diseases, lupus nephritis, immune complex renal diseases, glomerulopathies, nephritis, toxic nephropathy, obstructive uropathies and erectile dysfunction in a mammal comprising administering an effective amount of a PDE5 inhibitor.

9. A method for the treatment of a disease selected from the group of diseases consisting of Benign Prostate Hyperplasia (BPH), Bladder Outlet Obstruction (BOO) and Lower Urinary Tract Symptoms (LUTS) in a mammal comprising administering an effective amount of a PDE4 inhibitor.

10. A method for the treatment of a disease selected from the group of diseases consisting of Benign Prostate Hyperplasia (BPH), Bladder Outlet Obstruction (BOO) and Lower Urinary Tract Symptoms (LUTS) in a mammal comprising administering an effective amount of a combination of at least one PDE4 inhibitor and at least one PDE5 inhibitor.

11. A method for the treatment of a disease selected from the group of diseases consisting of Benign Prostate Hyperplasia (BPH), Bladder Outlet Obstruction (BOO) and Lower Urinary Tract Symptoms (LUTS) in a mammal comprising administering an effective amount of an agent which is an inhibitor of a PDE4 polypeptide and a PDE5 polypeptide.

12. A method for the treatment of a disease selected from the group of diseases consisting of Benign Prostate Hyperplasia (BPH), Bladder Outlet Obstruction (BOO) and Lower Urinary Tract Symptoms (LUTS), genitourinary disorders comprising neurogenic bladder syndrome (OAB) and (IC), urinary incontinence (UI), pelvic pain, benign and malign disorders of the organs constituting the genitourinary system of female and male, renal diseases, immunologically mediated renal diseases, lupus nephritis, immune complex renal diseases, glomerulopathies, nephritis, toxic nephropathy, obstructive uropathies and erectile dysfunction in a mammal comprising administering an effective amount of a PDE5 inhibitor selected from the group of PDE-5 Inhibitors consisting of Vardenafil, Sildenafil, Tadalafil, Udenafil, Dasantafil, Avanafil, SLx2101 and LAS34179.

13. A method for the treatment of a disease selected from the group of diseases consisting of Benign Prostate Hyperplasia (BPH), Bladder Outlet Obstruction (BOO) and Lower Urinary Tract Symptoms (LUTS) in a mammal comprising administering an effective amount of a combination of at least one PDE4 inhibitor selected from the group of PDE-4 inhibitors consisting of Roflumilast, Cilomilast and Piclamilast and at least one PDE-5 inhibitor selected from the group of PDE-5 inhibitors consisting of Vardenafil, Sildenafil and Tadalafil.

14. A pharmaceutical composition containing at least one compound selected from the group Vardenafil, Sildenafil, Tadalafil, Udenafil, Dasantafil, Avanafil, SLx2101 and LAS34179 or a salt, a hydrate or a hydrate of a salt thereof, for the treatment of a disease selected from the group of diseases consisting of Benign Prostate Hyperplasia (BPH), Bladder Outlet Obstruction (BOO), Lower Urinary Tract Symptoms (LUTS), genitourinary disorders comprising neurogenic bladder syndrome (OAB) and (IC), urinary incontinence (UI), pelvic pain, benign and malign disorders of the organs constituting the genitourinary system of female and male, renal diseases, immunologically mediated renal diseases, lupus nephritis, immune complex renal diseases, glomerulopathies, nephritis, toxic nephropathy and obstructive uropathies in a mammal.

15. A pharmaceutical composition for the treatment of neurogenic bladder, overactive bladder and interstitial cystitis containing at least one compound selected from the group Vardenafil, Sildenafil, Tadalafil, Udenafil, Dasantafil, Avanafil, SLx2101 and LAS34179 or a salt, a hydrate or a hydrate of a salt thereof.

16. A pharmaceutical composition for the treatment of neurogenic bladder, overactive bladder and interstitial cystitis containing Vardenafil or a salt, a hydrate or a hydrate of a salt thereof.

17. A method for the treatment of neurogenic bladder, overactive bladder and interstitial cystitis comprising administering an effective amount of at least one compound selected from the group Vardenafil, Sildenafil, Tadalafil, Udenafil, Dasantafil, Avanafil, SLx2101 and LAS34179 or a salt, a hydrate or a hydrate of a salt thereof.

18. A method for the treatment of neurogenic bladder, overactive bladder or interstitial cystitis comprising administering an effective amount of Vardenafil or a salt, hydrate, of a hydrate of a salt thereof.

19. The pharmaceutical composition of claim 7 wherein said urinary incontinence is mixed-, urge-, stress-, or overflow incontinence (MUI, UUI, SUI, OUI).

20. The pharmaceutical composition of claim 7 wherein said renal disease is acute or chronic renal failure.

21. The pharmaceutical composition of claim 7 wherein said immunologically mediated renal disease is renal transplant rejection.

22. The method of claim 8 wherein said urinary incontinence (UI) is mixed-, urge-, stress-, or overflow incontinence (MUI, UUI, SUI, OUI).

23. The method of claim 8 wherein said renal disease is acute or chronic renal failure.

24. The method of claim 8 wherein said immunologically mediated renal disease is renal transplant rejection.

25. The method of claim 12 wherein said urinary incontinence (UI) is mixed-, urge-, stress-, or overflow incontinence (MUI, UUI, SUI).

26. The method of claim 12 wherein said renal disease is acute or chronic renal failure.

27. The method of claim 12 wherein said immunologically mediated renal disease is renal transplant rejection.

28. The pharmaceutical composition of claim 14 wherein said urinary incontinence (UI) is mixed-, urge-, stress-, or overflow incontinence (MUI, UUI, SUI).

29. The pharmaceutical composition of claim 14 wherein said renal disease is acute or chronic renal failure.

30. The pharmaceutical composition of claim 14 wherein said immunologically mediated renal disease is renal transplant rejection.
